(19) United States
(12) Patent Application Publication
(10) Pub. No.: US 2017/0049473 A1
(43) Pub. Date: Feb. 23, 2017

(54) INSTRUMENT DOCKING PORTS FOR TRANS-ENDOSCOPIC AND LAPAROSCOPIC SURGERY ACCESS PORTS

(71) Applicant: Tanam Medical Inc., Victoria (CA)

(72) Inventors: Brad AMSON, Victoria (CA); Bao TANG, Victoria (CA)

(73) Assignee: Tanam Medical Inc., Victoria (CA)

(21) Appl. No.: 15/246,402

(22) Filed: Aug. 24, 2016

Related U.S. Application Data
(63) Continuation of application No. PCT/CA2015/050101, filed on Feb. 12, 2015.

(30) Foreign Application Priority Data
Feb. 24, 2014 (CA) 2843679

(51) Int. Cl. A61B 17/34; A61M 39/22; A61M 13/00

(52) U.S. Cl. CPC A61B 17/3423; A61B 17/3462; A61M 13/006; A61M 39/22

(57) ABSTRACT

Instrument docking ports and gas valve docking ports for sealingly engaging surgical access ports for use in transendoscopic and laparoscopic surgical procedures. The instrument docking ports comprise a cannula that defines a passageway for a surgical instrument, a rim outwardly extending around the circumference of the receiving end of the cannula that cooperates with one or more retaining protrusions outwardly extending about the external surface of the cannula to maintain the position of the cannula when installed for use. Disposed within the cannula is at least one sealing disc to allow insertion of a surgical instrument therethrough. The sealing disc sealingly engages with the surgical instrument when inserted and returns to a closed position when the surgical instrument is removed. The gas valve docking port has a gas ingress port and a gas egress port for controllable insufflation and desufflation of a target body cavity during a surgical procedure.

### TECHNICAL FIELD

The present disclosure pertains to the field of surgical devices and more specifically, to surgical access ports for use in minimally invasive surgical procedures such as laparoscopic and/or trans-endoscopic procedures.

### BACKGROUND

Minimally invasive surgery, such as endoscopic and/or laparoscopic procedures, is a type of surgery performed through one or more small incisions in a patient's body, usually less than an inch in dimension. Some advantages of minimally invasive surgery are that patients experience reduced physical and physiological trauma, much smaller scarring, and faster post-surgery recovery times.

The single port approach to minimally invasive surgery, whether that be through a single incision or via a natural orifice such as the oral cavity, nasal passages, the navel, the vagina, and the anus, for example, provides the benefit of smaller and fewer body cavity incisions. Single-port procedures, however, require several instruments to be inserted and manipulated through a single entry into a tissue tract, thereby, limiting the freedom of movement for manipulating the multiple instruments inserted through a single port.

Access ports are commonly used in such minimally invasive surgical procedures to facilitate the introduction and manipulation of multiple instruments and equipment into a tissue tract through an incision or a natural orifice. The access ports are used to protect adjacent tissues from potential abrasion and/or tearing and/or incised damage caused by insertion and manipulation of the surgical instruments and equipment.

Some procedures further involve the use of insufflation gases to enlarge the area surrounding the target surgical site to create a larger, more accessible work area. In such procedures, access ports may be introduced into regions that require maintenance of the pressurized gas.

In Canadian Patent No. 2,778,976, the inventors described a surgical access port comprising an elongate cannula that can be fixed within a tissue tract to define a passageway for introduction of one or more surgical instruments. The cannula extends into an enclosed dome comprised of a self-sealing material through which the one or more surgical instruments can be directly introduced. Being of a self-sealing material, the dome is deformable without losing physical integrity and maintains a substantially gas tight seal with any instrument extending therethrough, as well as self-sealing upon removal of the instruments. The expanded diameter of the dome permits angular or pivotal ranges of movement for the instruments passing through the dome, thereby permitting wide triangulation of instruments inserted therethrough. As well, greater spacing apart of multiple instruments is made possible to facilitate precise manipulation of instruments during surgical procedures. The combination of these features offer the surgeon flexibility in determining the placement and positioning of instruments in the dome as well as flexibility in selecting the size of instruments to be inserted.

In use, instruments are initially inserted at the desired location through the dome of the access port before positioning the access port in the patient. Surgeons, however, will typically require instruments to be exchanged during the surgical procedure and will remove instruments from the dome in exchange for a different instrument. Often, multiple exchanges of instruments are required during a surgical procedure. The inventors have found that the removal and re-insertion of instruments through the dome of the access port while the access port is positioned in the patient can be challenging. In particular, as a sufficient amount of force is required to insert the instrument through the dome, accurate positioning of the instrument may be compromised. As well, excessive force may inadvertently be applied when inserting a replacement instrument which may introduce unnecessary risk to the patient.

### SUMMARY

The exemplary embodiments of the present disclosure pertain to instrument docking ports for sealably receiving therethrough endoscopic surgical instruments and devices. The exemplary embodiments of the present disclosure also relate to docking ports comprising gas valves for controllable insufflation and desufflation of an endoscopic or a laparoscopic surgical site with a selected gas. The exemplary instrument docking ports and gas valve docking ports are particularly suitable for cooperating with resilient surgical access ports such as those exemplified in Canadian Patent No. 2,778,976. The exemplary instrument docking ports and gas valve docking ports are useful for cooperation with such surgical access ports for the performance of trans-endoscopic and laparoscopic surgical procedures.

One exemplary embodiment of the present disclosure pertains to instrument docking ports comprising: (a) a cannula defining an internal passageway and having a receiving end through which at least one surgical instrument can be introduced and a proximal end for insertion into a surgical access port; (b) at least one sealing disc disposed within the cannula, said sealing disc having a slit to allow insertion of the surgical instrument therethrough, wherein the sealing disc sealingly engages with the surgical instrument when inserted and returns to a closed sealed position when the surgical instrument is removed; (c) an outwardly extending rim around the circumference about the receiving end of the cannula for abutting the outer surface of a surgical access port; and (d) optionally, one or more retaining protrusions outwardly extending about the external surface of the cannula interposed the outwardly extending rim and its proximal end, wherein the rim and retaining protrusions cooperate to retain and maintain the position of the cannula when installed into a surgical access port for use in a surgical procedure.

Another exemplary embodiment of the present disclosure pertains to docking ports comprising a cannula having a gas valve about its distal end and a proximal end for insertion into and through surgical access ports that comprise resilient materials. The gas valves are switchable between: (i) an ingress port engageable with an external supply of a gas exemplified by carbon dioxide (CO₂) for controllably insufflating an endoscopic or a laparoscopic surgery target area, and (ii) an egress port for venting i.e. desufflating insufflated gases from the endoscopic or laparoscopic surgery target area. The exemplary gas valve docking ports have an outwardly extending rim around the circumference of the cannula interposed the gas valve and its proximal end, for abutting the outer surface of a surgical access port. The exemplary gas valve docking ports may optionally have one or more retaining protrusions extending outwardly about the external surface of the cannula between the rim and the proximal end of the cannula.

Another embodiment of the present disclosure pertains to surgical access ports for sealably engaging and cooperating with one or more of the exemplary instrument docking ports and gas valve docking ports wherein the surgical access ports comprise: (a) a cylindrical body having a first end and a second end, wherein the first end is for insertion into a target site and defines a passageway for introduction of one or more surgical instruments; (b) at least one retainer outwardly extending circumferentially about the first end of the cylindrical body, wherein the retainer engages an inside surface of the target site when the first end of the cylindrical body is inserted; (c) a self-sealing dome extending from the second end of the cylindrical body, the dome comprising an expanded surface area, the expanded surface area comprising a self-sealing material; and (d) one or more docking ports as described herein inserted at one or more locations through the expanded surface area of the self-sealing dome, wherein the self-sealing dome sealingly engages with the one or more docking ports when introduced therethrough to sealably fix the one or more docking ports therein.

Another embodiment of the present disclosure pertains to use of the instrument docking ports and gas valve docking ports in combination with the surgical access port disclosed herein for performing minimally invasive surgical procedures.

Another embodiment of the present disclosure pertains to kits comprising: (a) one or more of the exemplary instrument docking ports disclosed herein; and (b) a plunger for introducing said one or more instrument docking ports into surgical access port that comprises a resilient material. The exemplary kits may optionally additionally comprise one or more of the exemplary gas valve docking ports disclosed herein. The exemplary plunger generally comprises: (i) an elongated shaft having a piercer at one end and a handle at the opposite end, wherein the piercer is sized to fit through an exemplary instrument docking port, for piercing a selected target site on the surgical access port said insertion site, and for forcing the instrument docking port into and through the target site by an application of force to the handle; and (ii) a flange extending outward about the shaft to retain the instrument docking port on the plunger shaft when the piercer is forced through the target site on the surgical access port whereby the instrument docking port is inserted into and through the surgical access port and fixed therein.

Another embodiment of the present disclosure pertains to kits for performing a minimally invasive surgical procedure. The kits comprise:
(a) one or more instrument docking ports disclosed herein;
(b) one or more of the gas valve docking ports disclosed herein;
(c) a surgical access port comprising resilient materials formed into: (i) a cylindrical body having a first end and a second end, wherein the first end is for insertion into a target surgery access site and defines a passageway for introduction of one or more surgical instruments; (ii) at least one retainer outwardly extending circumferentially about the first end of the cylindrical body, wherein the retainer engages an inside surface of the target site when the first end of the cylindrical body is inserted; and (iii) a self-sealing dome extending from the second end of the cylindrical body, the dome comprising an expanded surface area, the expanded surface area comprising a self-sealing material, wherein the self-sealing dome sealingly engages with the one or more instrument docking ports and gas valve docking ports when introduced therethrough so as to sealably engage and fix the one or more instrument docking ports and gas valve docking ports therein; and
(d) a plunger for introducing the one or more instrument docking ports and gas valve docking ports into the self-sealing dome, wherein the plunger comprises: (i) an elongated shaft having a piercer at one end and a handle at the opposite end, wherein the piercer is sized to fit through the docking port to pierce the self-sealing dome and force the docking port into the self-sealing dome when force is applied to the handle; and (ii) a flange extending from the shaft to retain the docking port on the shaft when the piercer is forced through the self-sealing dome whereby the docking port is inserted into the self-sealing dome and fixed therein.

### BRIEF DESCRIPTION OF THE DRAWINGS

These and other features of this disclosure will become more apparent in the following detailed description in which reference is made to the appended drawings.

FIG. 1 is a perspective view of exemplary instrument docking ports and an exemplary gas valve docking port according to embodiments of the present disclosure, installed into and through the dome of a surgical access port comprising a resilient material, shown in a partial cross-sectional side view;

FIGS. 2A-2D are perspective side views of exemplary docking ports showing the portions of their cannula that are insertable into and through surgical access ports, wherein FIGS. 2(A), 2(B), and 2(C) show exemplary alternative outward extending retaining means, while FIG. 2(D) shows an optional exemplary docking port cannula that does not have any outward extending retaining means;

FIG. 3A is a top end view of an exemplary docking port having a plurality of sealing discs disposed therein according to embodiments of the present disclosure, while FIGS. 3B and 3C are cross-sectional side views of the docking port shown in FIG. 3A with exemplary spacing for separation of the sealing discs within the docking port;

FIG. 4 is a view of an exemplary instrument docking port provided with a cap component for sealing the receiving end of the instrument docking port;

FIGS. 5(A), 5(B), and 5(C) are perspective side views illustrating the steps for using an exemplary plunger for installation of an exemplary instrument docking port into the dome of a surgical access port comprising a resilient material;

FIG. 6 is a perspective view of the exemplary instrument docking port installed into and through the dome of the surgical access port from FIG. 5, shown with a surgical instrument inserted therethrough; and

FIGS. 7(A), 7(B), 7(C) are side views of an exemplary gas valve docking port according to embodiments of the present disclosure wherein FIG. 7(A) shows the gas valve turned to a position to receive a flow of a gas for insufflating a laparoscopic surgery target location, FIG. 7(B) shows the gas valve turned to a position to prevent insufflation and desufflation of the laparoscopic surgery target location, and FIG. 7(C) shows the gas valve turned to a position to allow the desufflating egress of gas from the laparoscopic surgery target location.

### DETAILED DESCRIPTION

### Definitions

Unless defined otherwise, all technical and scientific terms used herein have the same meaning as commonly understood by one of ordinary skill in the art to which this disclosure belongs.

As used herein, the term “about” refers to an approximately +/−10% variation from a given value. It is to be understood that such a variation is always included in any given value provided herein, whether or not it is specifically referred to.

The terms “subject” and “patient” as used herein refer to an animal in need of treatment.

The term “animal” as used herein, includes, but is not limited to, mammals including humans.

The terms “surgical instrument(s)” and “instrument(s)” as used herein, refer to any medical instrument used in minimally invasive surgical procedures and can include, but is not limited to, standard hand instruments and articulating instruments.

The terms “minimally invasive surgery” and “minimally invasive surgical procedure(s)” as used herein, refer to surgery performed through one or more small incisions in a patient's body or through a natural orifice such as the oral cavity, nasal passages, the navel, the vagina, and the anus, and includes endoscopic and/or laparoscopic procedures.

The terms “target site” and “target cavity” as used herein, refer to the location in the patient's body where the minimally invasive surgical procedure is to occur and can include, but is not limited to, the abdominal cavity and the rectum.

The term “tissue tract” as used herein, refers to the region of tissue through which instruments must pass through to reach the target site or target cavity to conduct the minimally invasive surgical procedure.

The term “insertion site” as used herein, refers to a target location on a surgical access port for inserting therethrough and into a docking port of the present disclosure. Suitable surgical access ports are exemplified by the self-sealing dome of a surgical access port described herein and in Canadian Patent No. 2,778,976.

The terms “insufflation” and “insufflating” as used herein, mean the controlled introduction of a pressurized flow of a selected gas into a body cavity exemplified by the abdominal cavity, the rectum, the large intestine, among others.

The terms “desufflation” and “desufflating” as used herein, mean the controlled release of an insufflated gas from a body cavity into which the gas was previously introduced.

The exemplary instrument docking ports disclosed herein provide means for creating self-sealing openings in a variety of medical device applications. Specific embodiments described herein, for example, illustrate the use of the instrument docking ports for facilitating the self-sealing insertion for endoscopic surgical instruments into and through a surgical access port into a target surgery site within a body cavity. In such applications, the instrument docking ports can be inserted into a surgical access port to tailor the positioning of the surgical instruments to the surgeon's individual preference. The ease of insertion and removal of surgical instruments from the instrument docking ports disclosed herein enables surgeon to sequentially use multiple types of surgical instruments through two or three instrument docking ports during the performance of a surgical procedure thereby significantly reducing the clutter of surgical instruments that previously had to inserted all together at one time prior to commencing surgical procedures.

The exemplary instrument docking ports disclosed herein can be sized to accommodate a variety of different types and sizes of surgical instruments. As well, the instrument docking ports of the present disclosure can be sized to accommodate surgical instruments that are designed for larger-sized subjects as well as for juvenile/infant applications. The instrument docking ports of the present disclosure are, in this way, also adaptable to the individual patient.

The exemplary instrument docking ports disclosed herein may comprise one or more sealing discs through which surgical instruments are inserted. Specifically, the sealing discs sealingly engage the instrument when inserted and self-seal upon instrument removal, thus minimizing and/or eliminating leakage of fluids and gases from the surgical access port and enabling pressurization within a target surgery site to be maintained in procedures that require the injection and maintenance of insufflation gases. Furthermore, the sealing discs disposed within the instrument docking ports allow insertion of surgical instruments through the instrument docking ports of the present disclosure without impeding the motion of, or increasing the friction on, the surgical instrument. In this way, withdrawal or exchange of instruments from the instrument docking ports can be achieved with ease and without risk of trauma to the patient.

### Instrument Docking Ports

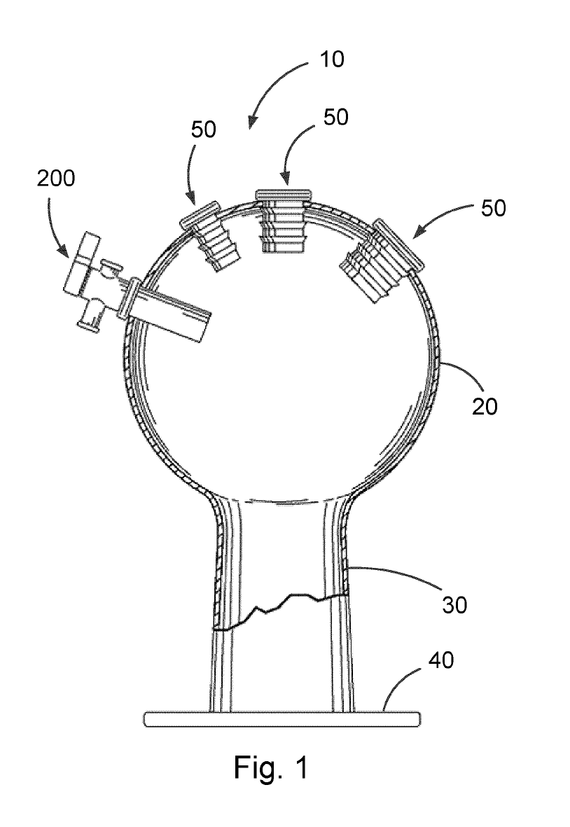

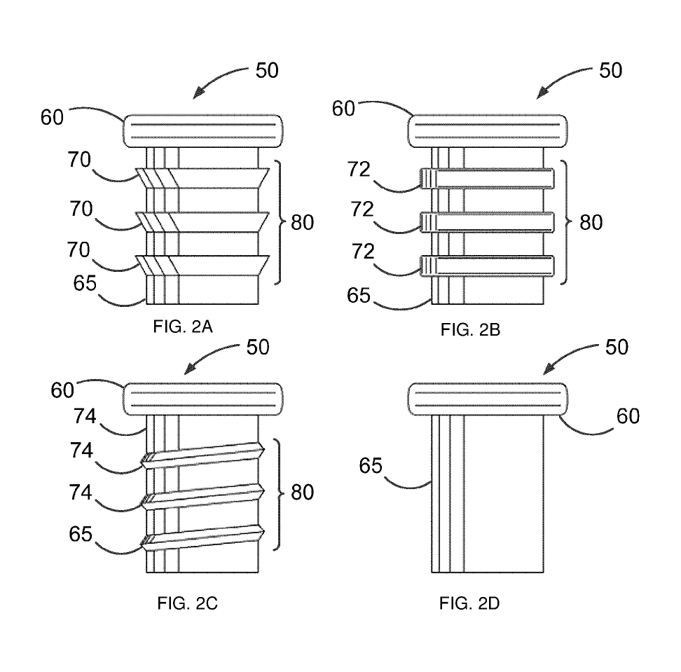

Referring now to the drawings in which like reference numerals identify identical or substantially similar parts throughout the several views, FIGS. 2(A), 2(B), 2(C) and 2(D) illustrate perspective side views of some exemplary instrument docking ports of the present disclosure, showing the portions of their cannula that are insertable into and through surgical access ports. Instrument docking port 50 includes an elongate cannula 65 that defines an internal passageway. The cannula 65 has a receiving end at its distal end through which at least one surgical instrument can be introduced to pass through the internal passageway of the cannula 65. A rim 60 extends outwardly around the circumference of the receiving end of the cannula 65. FIGS. 2(A), 2(B), 2(C) show alternative embodiments of pluralities of outwardly extending protrusions (70, 72, 74 respectively) from the cannula 65, to form a retaining means 80. Together, the rim 60 and retaining means 80 cooperate to securely maintain the position of the instrument docking port 50 when installed into a surgical access port (e.g., item 20 in FIG. 1) for use. This is further illustrated in FIG. 1 which shows embodiments of the instrument docking port 50 of the present disclosure installed into and through a surgical access port 10 (for example, the surgical access ports disclosed in Canadian Patent No. 2,778,976). As illustrated, the rim 60 ensures that the receiving end of the cannula 80 abuts and retained on the outer surface of the access port 10 after installation. The retaining means 80 shown in FIGS. 2(A), 2(B), 2(C) similarly ensure that the cannula 65 is internally retained after installation into and through the surgical access port 10. Together the rim 60 and the retaining means 80 fix the instrument docking port 50 in position and maintain the position of the instrument docking port 50 after installation into and through the surgical access port 10. As illustrated in FIGS. 2(A), 2(B), and 2(C), the retaining protrusions 70, 72, 74 can be in the form of retaining threads, for example a buttress thread form 70, a square thread form 72, or a trapezoidal thread form 74. The number of threads, the pitch, and/or the angle of the threads can vary depending on the application. It is further contemplated that the retaining protrusions can also take a variety of alternative forms, examples of which include without limitation, barbs or ribs. Alternatively, for some applications, retaining means are not necessary and accordingly, the instrument docking ports of the present disclosure may simply comprise a cannula 65 and a rim 60 as shown in FIG. 2(D).

The dimensions (i.e., the diameter and length) of the instrument docking ports 50 can be varied for different uses and applications. As illustrated in FIG. 1, the instrument docking ports 50 of the present disclosure can be sized to accommodate the insertion of different types and sizes of endoscopic and laparoscopic surgical instruments to accommodate different types of surgical procedures, the types of target sites, and patient sizes ranging from juvenile subjects to adult subjects. As well, the instrument docking ports of the present disclosure can be sized to accommodate surgical procedures in human subjects or alternatively, animal subjects for veterinary surgical use. For example, the instrument docking ports 50 may have a length of about 5 mm, 7 mm, 10 mm, 12 mm, 14 mm, 16 mm, 18 mm, 20 mm, 22 mm, 24 mm, 26 mm, 28 mm, 30 mm, or any length therebetween. For other applications, the instrument docking ports 50 may have a length greater than about 30 mm. Similarly, the diameter of the aperture 50 can vary depending on the intended use. For example, the instrument docking ports 50 can have an inner diameter of at least about 5 mm. In some embodiments, the instrument docking ports 50 may have an inner diameter of about 5 mm, 7 mm, 10 mm, 12 mm, 15 mm, or any diameter therebetween.

In order to secure the instrument docking ports 50 to surgical access ports at the insertion sites, the outer diameter of the rim 60 and the retainer protrusions 70, 72, 74, respectively, must be larger than the external diameter of the cannula 65. In some embodiments, the outer diameter of the rim 60 and retainer protrusions 70, 72, 74 can be independently about 0.5% to about 50% larger than the outer diameter of the cannula 65. The diameter of the rim 60 and individual retainer protrusions 70, 72, 74 may be the same or may differ from one another. Alternatively, the retainer protrusions may be in the form of an outwardly extending thread winding from about the proximal end of the cannula 65 toward the rim 60.

The specific dimensions of the exemplary instrument docking ports can be selected as needed for particular applications. Specifically, it is envisioned that a wide variety of sizes will be available to a user to enable the user to select the most appropriately dimensioned aperture for the patient and procedure at hand. The overall dimensions of the exemplary instrument docking ports disclosed herein can vary. Also, the relative dimensions of the cannula, rim, and retainer protrusions of the aperture can vary. The cannula 65, rim 60 and retainer protrusions 70, 72, 74 can be a single integrated unit and may be made from any suitable materials, such as stainless steel, titanium, thermoplastics, and the like.

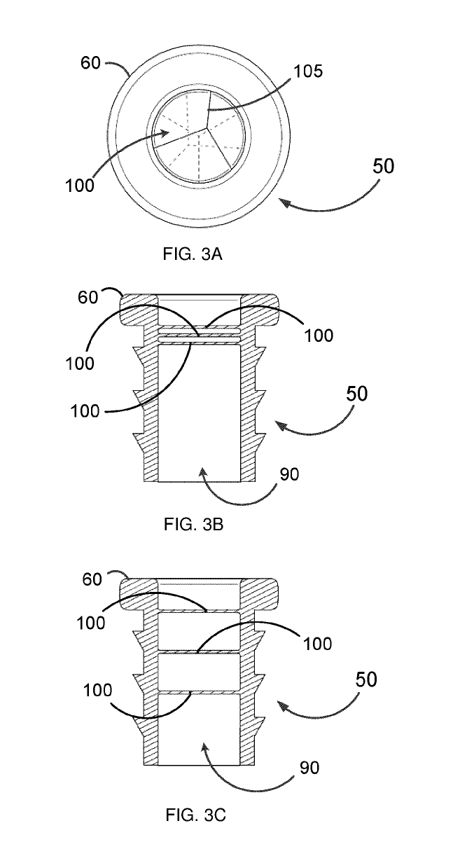

Disposed within the internal passageway 90 of the cannula 65, as shown in FIGS. 3(A), 3(B), and 3(C), is at least one sealing disc 100 designed to allow insertion of a surgical instrument therethrough and for sealingly engaging the surgical instrument after its insertion. Upon withdrawal of the surgical instrument from the instrument docking port 50, the sealing disc 100 returns to a closed sealed position. In this way, particularly in procedures where insufflation gases are employed for example, the sealing disc(s) 100 permit pneumoperitoneum to be maintained during multiple instrument exchanges and substantially prevent unintentional loss of pneumoperitoneum.

In some exemplary embodiments, the instrument docking port 50 comprises a single sealing disc 100. In other exemplary embodiments, the instrument docking port 50 comprises a plurality of sealing discs 100. In some exemplary embodiments, the instrument docking port 50 comprises three sealing discs 100. In other exemplary embodiments, the instrument docking port 50 may comprise up to ten sealing discs 100 disposed within the cannula 65. Each sealing disc 100 is approximately circular, contains three slits 105, and is sized to securely fit within the cannula 65. The thickness of the sealing disc(s) 100 is dependent on the length of the cannula 65 and the number of sealing disc(s) 100 disposed therein. In some embodiments, the thickness of the sealing disc 100 is between about 0.5 mm to about 3 mm. Sealing disc(s) 100 may be made from any appropriate material, such as rubber or any suitable elastomer, but is most preferably made from a silicone rubber, such as Silastice silicone rubber sold by Dow Corning Corporation, Midland, Mich.

The slits 105 in each sealing disc 100 are located slightly off center of the sealing disc 100 and extends entirely through the thickness of the sealing disc 100. The slits 105 are preferably made such that the sealing disc 100 forms resilient flaps (FIG. 3A) that deform inwardly without impeding the motion of, or increasing the friction on, a surgical instrument being inserted therein. When the surgeon wishes to withdraw or exchange instruments, the surgical instrument is withdrawn from the instrument docking port 50 and the slits 105 return to their original closed position, thereby sealing the instrument docking port 50.

As further shown in FIG. 3A, in embodiments comprising a plurality of sealing discs 100, each sealing disc 100 is disposed within the instrument docking port 50 such that the respective slits 105 are offset from each other to ensure a leak-proof seal. In such embodiments, the sealing discs 100 can be spaced at an appropriate distance apart from each other within the length of the passageway 90 to maintain a leak-proof seal.

It is optional for other types of gas-tight seals known to those skilled in these arts for forming gas-tight seals with surgical instruments passed through the seals, to be integrally disposed within the exemplary instrument docking port 50 of the present disclosure. For example, suitable is a gas-tight seal exemplified by the disclosure in U.S. Reissued Pat. No. 42,379 comprising a seal body, an instrument seal, and a laterally-compliant seal mounting device wherein the seal body includes a bore through which the surgical instrument is passed. Also suitable is the exemplary plurality of coaxial sealing elements adapted for foiming a seal with either a large or small surgical instrument, disclosed in U.S. Pat. No. 8,147,458. The plurality of coaxial sealing elements may comprise a first seal assembly, a second seal assembly, and a third seal assembly preferably mounted in the present exemplary instrument docking port 50 in a coaxial relationship. The first seal assembly comprises a plurality of protectors and a first seal element comprising a radially stretchable septum coupled to the protectors wherein at least one of the protectors comprises a proximal bump configured to contact and shield a large instrument from contracting the first seal element. The second seal assembly comprises a guide and a second seal element coupled to the guide, for forming a second seal with a large surgical instrument. The third seal assembly comprises a third seal element defining a plenum in which at least a portion of the second seal assembly is disposed, and preferably comprises a zero-seal exemplified by a duck-bill seal. Also suitable is a pleated trocar shield comprising a seal assembly exemplified in U.S. Pat. No. 8,257,317 wherein the seal assembly comprises a first zero seal for sealing the cannula 65 of the present instrument docking port 50 in the absence of a surgical instrument extending therethrough. The seal assembly also comprises a second instrument seal for sealing the cannula 65 in the presence of a surgical instrument extending therethrough, while the third seal is a trocar shield for protecting the first seal and the second seal from damage as a surgical instrument is advanced therethrough. The trocar shield comprises an open proximal end and a tapered distal end comprising a plurality of longitudinal pleats converging in an opening. Also suitable is the exemplary seal comprising a gel material as disclosed in U.S. Pat. No. 8,241,251 wherein the seal includes inner first seal portions defining an access channel for forming a sealing relationship with a surgical instrument advanced therethrough and a second material having a hardness greater than the hardness of the gel material to stabilize the first inner seal portions and to provide a substantial sealed relationship with the surgical instrument. Also suitable is the exemplary seal member disclosed in U.S. Pat. No. 8,206,358 wherein the seal member has a substantially annular member partially embedded within the seal member. The annular member is rigid relative to the seal member and defines an opening for permitting the passage therethrough of a surgical instrument while the internal seal surfaces of the seal member sealingly engage the surgical instrument, whereby the annular member minimizes the offset manipulation of the surgical instrument relative to the longitudinal plane of the present instrument docking port 50. It is to be noted that if so desired, combinations of the above-noted exemplary prior art gas-tight seals may be integrally engaged in series along the length of the cannula 65 of the present instrument docking port 50.

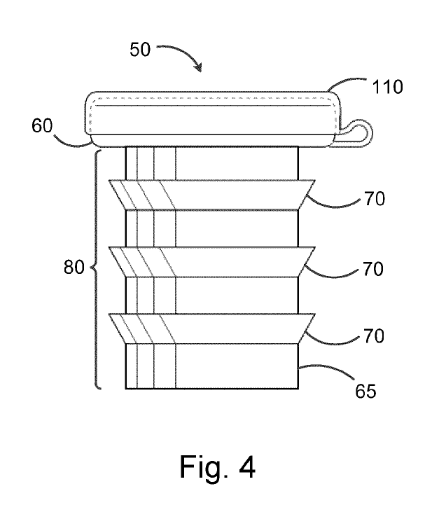

In certain embodiments, the exemplary instrument docking port 50 of the present disclosure may further include a cap 110 (FIG. 4) which can be used to close the receiving end of the instrument docking port 50 and prevent possible leakage of fluid or escape of pressure from the target site when the particular instrument docking port 50 is not in use, i.e., when a surgical instrument has been withdrawn.

Installation of the Instrument Docking Port(s) into a Surgical Access Port

The exemplary instrument docking ports 50 of the present disclosure can be installed to provide access, particularly sealable access, to endoscopic and laparatoscopic surgical instruments into a surgical access port. In a preferred embodiment, the instrument docking port 50 is suitable for use with the surgical access port described in Canadian Patent No. 2,778,976 as illustrated in FIG. 1. The surgical access port 10 comprises a cylindrical body 30 having at least one retainer 40 outwardly extending circumferentially about one end to secure the access port 10 in the target site. The opposite end of the cylindrical body 30 extends into a dome 20 comprising a self-sealing material which provides sealing engagement with instrument docking ports 50 that have been installed therein. The self-sealing material of the dome 20 is pliable and resilient enough to allow the insertion of the apertures 50 of the present disclosure and can comprise, for example, rubber, synthetic rubber, silicone, ethylene propylene diene monomer (EPDM), ethylene-propylene copolymer (EP rubber), polyisoprene, polybutadiene, polyurethane, styrene-butadiene, ethylene vinyl acetate (EVA), polychloroprene also referred to as neoprene, perfluoroelastomer exemplified by KALREZ® (KALREZ is a registered trademark of E.I. Du Pont de Nemours and Co. Corp., Wilmington, Del., USA), and the like, or any combination thereof.

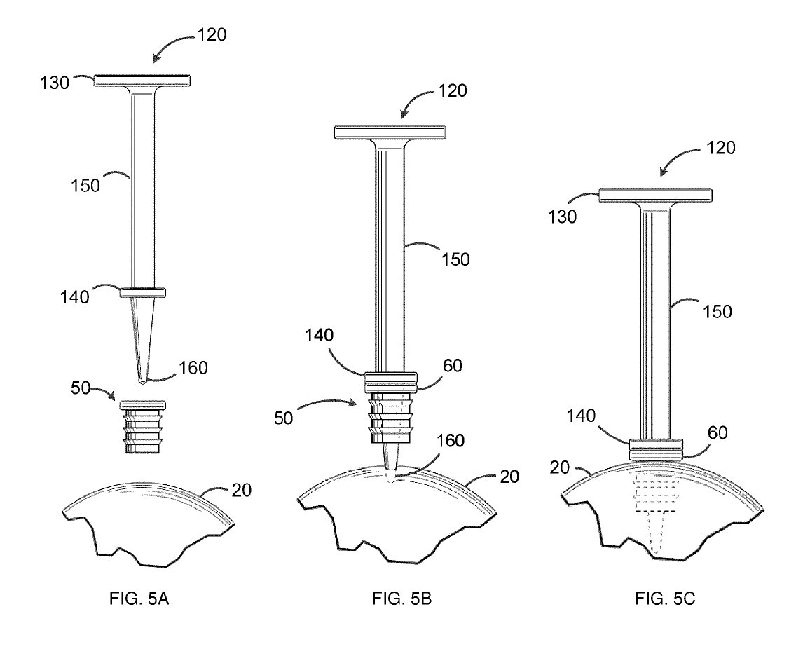

Referring to FIGS. 5(A), 5(B), and 5(C), installation of an instrument docking port 50 into the self-sealing dome 20 of the surgical access port 10, for example, is achieved by forcibly inserting the instrument docking port 50 through the surface of the dome 20 until the rim 60 of the instrument docking port 50 rests on the exterior surface of the dome 20 and the retaining protrusions have been inserted through the surface of the dome 20 and into the interior of the dome 20.

As illustrated in FIGS. 5(A), 5(B), and 5(C), the exemplary instrument docking port 50 of the present disclosure can be installed with the aid of a plunger 120. The plunger 120 comprises an elongated shaft 150 having a piercer 160 at one end that is sized to fit through the instrument docking port 50 within the internal passageway. The shaft 150 extends into a handle 130 at the opposite end. A flange 140 is disposed along the shaft 150 above the piercer 160 to position an instrument docking port 50 for installation into the surgical access port 20. As shown in FIG. 5(B), once the instrument docking port 50 is mounted onto the shaft 50, force is applied to the handle 120 to pierce the surface of the surgical access port 20 and force the instrument docking port 50 therethrough at the desired insertion site. Once the rim 60 abuts the external outer surface of the surgical access port 20 (FIG. 5(C)), the instrument docking port 50 is sealingly fixed into place and the plunger 120 can be removed.

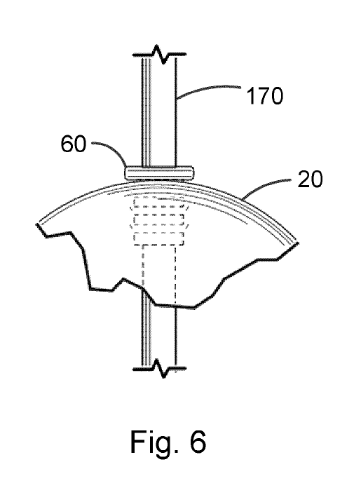

After the instrument docking ports 50 are installed at the desired insertion site on a surgical access port 20, surgical instruments 170 can be repeatedly inserted and withdrawn and/or exchanged through the installed docking instrument docking port 50 (FIG. 6). Should removal and/or repositioning of the instrument docking port 50 be desired, the instrument docking port 50 can simply be forcibly pulled from the surgical access port 20 which will then self-seal at the site of insertion.

### Gas Valve Docking Ports

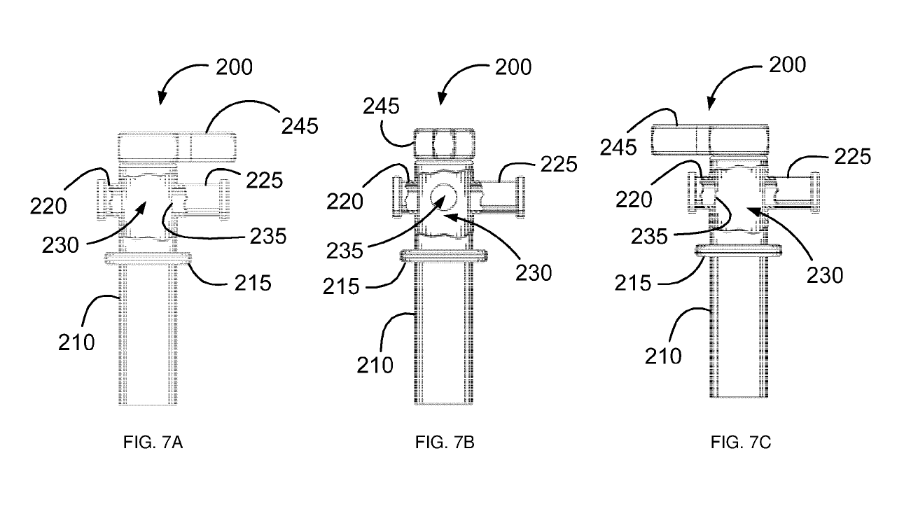

Some exemplary embodiments of the present disclosure pertain to docking ports for sealably engaging and communicating with surgical access ports, wherein the distal ends of the docking ports comprise a gas valve switchable between an ingressing gas port, an egressing gas port, and a closed position interposed the ingressing gas port and the egressing gas port. An exemplary gas port 200 of the present disclosure is shown in FIGS. 7(A)-7(C) and generally comprises an outer sleeve 210 with a first sideway extending port 225 about the distal end of the sleeve 210 for engaging a supply line from a pressurized gas source (referred to hereafter as an “ingress port”) and a second sideway extending port 220 (referred to hereafter as an “egress port”) opposite the first port 225. The sleeve 210 has an outwardly extending retaining rim 215 encircling the circumference of the sleeve 210. Inside sleeve 210 is a conduit 230 having: (i) a handle 245 engaged with the distal end of the conduit 230, and (ii) an orifice 235 positioned to communicate with ingress port 225 and egress port 220. The handle 245 preferably extends outward from the conduit 230 over the orifice 235.

As illustrated in FIG. 1, the exemplary gas valve port 200 can be installed into a suitable surgical access port such as those exemplified in Canadian Patent No. 2,778,976 by inserting the proximal end of the sleeve 210 into and through the dome 20 of the surgical access port 10. It is optional if so desired, to provide as series of retaining protrusions (exemplified by protrusions 70, 72, 74 in FIGS. 3(A), 3(B), 3(C)) extending outward from the exterior circumference of the sleeve 210. After installation of the gas valve docking port 200 into a surgical access port 10 along with one or more instrument docking ports 50, and the surgical access port 10 is installed into a target access point in a mammalian subject for performance of a endoscopic or laparoscopic surgical procedure, a gas line from a source of pressurized gas can be engaged with the ingress port 225 of the gas valve port 200 after which, the handle 245 is turned to partially or completely expose the orifice 235 to the ingress port 225 (FIG. 7(A)) for controllable insufflation of the target surgery cavity within the mammalian subject. After sufficient insufflation of the target cavity has been achieved, the handle 245 is turned so that the orifice 235 is position in between the ingress port 225 and the egress port 220 (FIG. 7(B)). After the surgical procedure has been completed, the handle 245 is turned to partially or completely expose the orifice 235 to the egress port 220 (FIG. 7(C)) for controllable desufflation of the target surgery cavity. It is optional to engage the exgress port 220 with a gas line for conveying the desufflated gas away into a suitable receptacle or venting device.

### Kits

Some exemplary embodiments of the present disclosure pertain to kits that comprise a plurality of the exemplary instrument docking ports of the present disclosure in a range of sizes along with a suitable sized plunger or plungers for installing the instrument docking ports. The kits may additionally comprise one or more gas valve docking ports.

Some exemplary embodiments of the present disclosure pertain to kits for performing a minimally invasive surgical procedure wherein the kits comprise a plurality of the exemplary instrument docking ports of the present disclosure in a range of sizes, one or more of the exemplary gas valve docking ports, a suitable sized plunger or plungers for installing the instrument docking ports, and a surgical access port comprising a resilient material such as described herein, and also, exemplified in Canadian Patent No. 2,778,976. The kits may optionally contain instructions or directions listing the methods for installation of the instrument docking ports and/or the gas valve docking ports. The kits may also comprise one or more endoscopic or laparoscopic surgical instruments for performing a surgical procedure. Suitable surgical instruments are exemplified by trocars, scissors, graspers, dissectors, fixation forceps, dissecting forceps, cauterizing electrodes, suturing instruments, and the like.

1. An instrument docking port for sealingly engaging an endoscopic or laparoscopic surgical access port, the instrument docking port comprising:
a cannula defining an internal passageway, said cannula having a distal end and a proximal end for receiving therethrough a surgical instrument;
at least one self-sealing disc disposed within the cannula for receiving therethrough the surgical instrument wherein the self-sealing disc sealingly engages the surgical instrument when it is inserted through the distal end of the cannula, and returns to a self-sealed closed position when the surgical instrument is removed from the cannula; and
a rim outwardly extending around the circumference of the distal end of the cannula.

2. The instrument docking port of claim 1, wherein the at least one self-sealing disc comprises three overlapping flaps.

3. The instrument docking port of claim 1, comprising between 2 and 10 self-sealing discs.

4. The instrument docking port of claim 1, having a seal assembly disposed within the cannula, said seal assembly comprising a seal body having a bore for receiving a surgical instrument therethrough, an instrument seal for sealingly engaging the surgical instrument, and a laterally-compliant seal mounting device.

5. The instrument docking port of claim 1, having three seal assemblies disposed within the cannula, wherein (i) the first seal assembly comprises a plurality of protectors and a first seal element comprising a radially stretchable septum coupled to the protectors wherein at least one of the protectors comprises a proximal bump configured to contact and shield a large instrument from contracting the first seal element, (ii) the second seal assembly comprises a guide and a second seal element coupled to the guide, for forming a second seal with the large surgical instrument, and (iii) the third seal assembly comprises a third seal element defining a plenum in which at least a portion of the second seal assembly is disposed.

6. The instrument docking port of claim 1, having a seal assembly disposed within the cannula, wherein the seal assembly comprises (i) a first zero seal for sealing the cannula in the absence of a surgical instrument extending therethrough, (ii) a second instrument seal for sealing the cannula in the presence of a surgical instrument extending therethrough, and (iii) a third seal for protecting the first seal and the second seal from damage as a surgical instrument is advanced therethrough, said third seal comprising an open proximal end and a tapered distal end comprising a plurality of longitudinal pleats converging in an opening.

7. The instrument docking port of claim 1, having a seal assembly disposed within the cannula, wherein the seal assembly comprises (i) inner first seal portions defining an access channel for forming a sealing relationship with a surgical instrument advanced therethrough, said inner first seal portions comprising a gel material, and (ii) a second material having a hardness greater than the hardness of the gel material for stabilizing the first inner seal portions and to provide a substantial sealed relationship with the surgical instrument.

8. The instrument docking port of claim 1, having a seal member disposed within the cannula, wherein the seal member has a substantially annular member partially embedded within the seal member, said annular member rigid relative to the seal member, said annular member defining an opening for permitting the passage therethrough of a surgical instrument while the internal seal surfaces of the seal member sealingly engage the surgical instrument.

9. The instrument docking port of claim 1, wherein the cannula has a retaining protrusion extending outward around the external circumference of the cannula in the form of a buttress thread winding from a proximal end of the cannula toward the rim.

10. The instrument docking port of claim 1, additionally having a plurality of spaced-apart circular protrusions extending outward from the outer circumference of the cannula, each of said protrusions having a buttress form.

11. The instrument docking port of claim 1, additionally having a plurality of spaced-apart circular protrusions extending outward from the outer circumference of the cannula, each of said protrusions having a square form.

12. The instrument docking port of claim 1, additionally having a plurality of spaced-apart circular protrusions extending outward from the outer circumference of the outer sleeve, each of said protrusions having a trapezoidal form.

13. The instrument docking port of claim 1, wherein the cannula has a diameter of at least 5 mm.

14. The instrument docking port of claim 1, wherein the cannula has a diameter of between 5 mm and 15 mm.

15. The instrument docking port of claim 1, wherein the cannula has a length of between 5 mm and 10 mm.

16. The instrument docking port of claim 1, wherein the cannula has a length of between 10 mm and 20 mm.

17. The instrument docking port of claim 1, wherein the cannula has a length of between 20 mm and 30 mm.

18. The instrument docking port of claim 1, wherein the cannula has a length of at least 30 mm.

19. The instrument docking port of claim 1, additionally comprising a removable cap for covering the distal end of the cannula.

20. A kit of parts comprising at least one instrument docking port of claim 1.

21. The kit of parts of claim 20, additionally comprising a plunger for installation of the instrument docking port into the endoscopic or laparoscopic surgical access port.

22. The kit of parts of claim 20, additionally comprising a gas valve docking port comprising (i) an outer sleeve having a gas ingress port extending outward about the distal end of the sleeve and a gas egress port extending outward from the sleeve opposite the gas ingress port, and (ii) a conduit rotatably housed within the sleeve, said conduit engaged with a handle at a distal end of the conduit, said conduit having an orifice rotatably communicable with the gas ingress port and the gas egress port.

23. The kit of parts of claim 22, wherein the gas docking port has a retaining protrusion extending outward around the external circumference of the outer sleeve.

24. The kit of parts of claim 20, additionally comprising a surgical access port, said surgical access port comprising:
a cylindrical body having a first end and a second end, wherein the first end is for insertion into a target site and defines a passageway for introduction of one or more surgical instruments;
at least one retainer outwardly extending circumferentially about the first end of the cylindrical body, wherein the retainer engages an inside surface of the target site when the first end of the cylindrical body is inserted; and
a self-sealing dome extending from the second end of the cylindrical body, the dome comprising an expanded surface area, the expanded surface area comprising a self-sealing material.

25. A gas valve docking port for sealingly engaging an endoscopic surgical access port or a laparoscopic surgical access port, the gas valve docking port comprising:
an outer sleeve having (i) a gas ingress port extending outward about a distal end of the sleeve, (ii) a gas egress port extending outward from the sleeve opposite the gas ingress port, and (iii) a rim outwardly extending around the circumference of the outer sleeve adjacent about the gas ingress port and the gas egress port; and
a conduit rotatably housed within the sleeve, said conduit engaged with a handle at a distal end of the conduit, said conduit having an orifice rotatably communicable with the gas ingress port and the gas egress port.

26. The gas valve docking port of claim 25, wherein the outer sleeve has a retaining protrusion extending outward around the external circumference of the outer sleeve in the form of a buttress thread winding from a proximal end of the outer sleeve toward the rim.

27. The gas valve docking port of claim 26, additionally having a plurality of spaced-apart circular protrusions extending outward from the outer circumference of the outer sleeve, each of said protrusions having a buttress form.

28. The gas valve docking port of claim 26, additionally having a plurality of spaced-apart circular protrusions extending outward from the outer circumference of the outer sleeve, each of said protrusions having a square form.

29. The gas valve docking port of claim 26, additionally having a plurality of spaced-apart circular protrusions extending outward from the outer circumference of the outer sleeve, each of said protrusions having a trapezoidal form.
